(19) United States
(12) Patent Application Publication
(10) Pub. No.: US 2022/0295846 A1
(43) Pub. Date: Sep. 22, 2022

(54) TASTE MODIFYING INGREDIENT DERIVED FROM RICE PROTEIN

(71) Applicant: Givaudan SA, VERNIER (CH)

(72) Inventors: Yili WANG, Mason, OH (US); Moises GALANO, Sao Paulo (BR); Jing JIANG, Cincinnati, OH (US); Pablo Victor KRAWEC, Mason, OH (US); Jenifer AUGELLI, Sharonville, OH (US)

(21) Appl. No.: 17/639,085

(22) PCT Filed: Sep. 9, 2020
(86) PCT No.: PCT/EP2020/075151

§ 371 Date: Feb. 28, 2022

Related U.S. Application Data
(60) Provisional application No. 62/902,965, filed on Sep. 19, 2019.

(51) Int. Cl. A23L 27/21; A23J 3/34; A23L 2/56; A23L 2/60; A23L 2/66; A23L 27/00; A23L 27/30

(52) U.S. Cl. CPC A23L 27/21; A23J 3/346; A23L 2/56; A23L 2/60; A23L 2/66; A23L 27/84; A23L 27/88; A23L 27/34; A23L 27/36; A23V 2002/00

(57) ABSTRACT

A method for making a taste modifying ingredient is provided. The method includes a) subjecting a rice protein to enzymatic hydrolysis to obtain a reaction mixture; b) separating the reaction mixture to obtain a supernatant; and c) recovering the supernatant of the reaction mixture.

### TECHNICAL FIELD

The present disclosure relates to methods for making taste modifying ingredients using rice protein and the taste modifying ingredients made by the methods. More particularly, the present disclosure relates to flavour compositions and consumables comprising the taste modifying ingredients and the uses of the taste modifying ingredients in consumables, for example to inhibit the degradation of sweeteners and/or sweetness enhancers, improve mouthfeel of consumables and/or mask off-notes of consumables and/or improve sweetness of consumables.

### BACKGROUND

Compounds for modifying the taste of consumable products, that is, products taken orally either for ingestion or spitting out, such as foodstuffs, beverages, confectionery, oral care products and the like are widely used. They do not themselves add flavour to the consumable, but they provide desirable ancillary benefits, such as enhanced mouthfeel, or masking undesirable characteristics of other ingredients, such as the bitter aftertaste associated with some sweeteners used in place of sugar in dietary products. Further, food and beverage formulations need to be produced with stable ingredients, particularly when such formulations or products are subject to periods of shelf life. Degradation of food and beverage formulations can be caused by many factors such as temperature (heat), pH, light and other factors.

By “mouthfeel” is meant the physical sensation(s) in the mouth caused by the presence therein of a consumable product such as a foodstuff or a beverage. A desirable mouthfeel is often typified by smoothness (lack of roughness and abrasion) and creaminess. Unfortunately, the desirable effect is often a feature of fatty foods, such as dairy products, the intake of which should be reduced for many people for health and dietary reasons.

In particular, there remains a need to provide taste modifying ingredients which are natural and/or suitable for vegans, i.e. “cleaner label”. Novel taste modifying ingredients and methods for making the taste modifying ingredients are therefore provided by the present disclosure. In addition, there remains a need to provide taste modifying ingredients that complement flavours of edible compositions in which they are incorporated in, in order to accentuate flavours and mouthfeel of said compositions, rather than exert their own particular taste characteristics; and so lend themselves to a very broad spectrum of use across a wide range of consumable categories.

### SUMMARY

In one illustrative embodiment, a process for making a taste modifying ingredient comprises the steps of: a) subjecting a rice protein to enzymatic hydrolysis to obtain a reaction mixture; b) separating the reaction mixture to obtain a supernatant; and c) recovering the supernatant of the reaction mixture.

In another illustrative embodiment, a flavour composition comprises a characterizing flavour; and a taste modifying composition comprising a rice protein isolate. In yet another illustrative embodiment, a beverage comprises a flavour composition comprising a characterizing flavour and a taste modifying composition; and one or more sweeteners. The taste modifying composition comprises a rice protein isolate.

These and other features, aspects and advantages of specific embodiments will become evident to those skilled in the art from a reading of the present disclosure.

### DETAILED DESCRIPTION

The following text sets forth a broad description of numerous different embodiments of the present disclosure. The description is to be construed as exemplary only and does not describe every possible embodiment since describing every possible embodiment would be impractical, if not impossible. It will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims. All publications and patents cited herein are incorporated herein by reference.

The present disclosure relates to the surprising finding that subjecting rice protein to enzymatic hydrolysis produces a product that can be used as a taste modifying ingredient, for example, to inhibit the degradation of sweeteners and/or sweetness enhancers, to improve the mouthfeel of a consumable, to mask off-notes of a consumable and/or to improve the sweetness of a consumable. In particular, the present disclosure relates to the surprising finding that the taste modifying ingredients described herein can be used to provide a natural and low-calorie oriented beverage with suppressed decrease in sweetness during storage. In other words, the taste modifying ingredients described herein can be used to enable retention/preservation of sweet intensity and sweetness overall quality of sweetness enhancers.

This finding was all the more surprising considering that when applicant tasted the compounds in dilute aqueous solution, they were either tasteless or they exhibited no inherent sweetness. As such, they appeared to be quite unsuitable for use in flavour applications. Only their combination with other flavour co-ingredients and the judicious selection of their usage levels was it possible to discover the remarkable organoleptic properties of these compounds. Their effect on edible compositions is quite unusual in that rather than exerting a characteristic flavour profile to a foodstuff or a beverage, they actually complement, lift or accentuate the sweet intensity and sweetness overall quality of the foods or beverages in which they are incorporated. Accordingly, the compounds of the present invention find utility in a broad spectrum of applications in the food and beverage industry.

In certain embodiments, the present disclosure discloses a method of inhibiting the degradation of sweeteners and/or sweetness enhancers contained in consumables by adding a degradation-inhibiting amount of one or more taste modifying ingredients to the consumables.

Sweetness enhancers are compounds that enhance the sweetness of carbohydrate sweeteners or high potency sweeteners thereby allowing the formulations of foods, beverages and other sweet edible formulations with less sweetener compared to equally sweet formulations not containing a sweetness enhancer. The benefits of sweetness enhancers include a lower sweetener cost and in the case of caloric carbohydrate sweeteners, a lower calorie food, beverage or other sweet edible formulation that maintains the carbohydrate sweet taste profile.

The present disclosure relates to a process for making a taste modifying ingredient comprising the steps of: a) subjecting a rice protein to enzymatic hydrolysis to obtain a reaction mixture; b) separating the reaction mixture to obtain a supernatant; and c) recovering the supernatant of the reaction mixture.

### Rice Protein

The term “rice protein” refers to a vegetarian protein isolate that is an alternative to the more common whey and soy protein. Rice protein may be derived from brown rice treated with enzymes that causes carbohydrates to separate from proteins. Rice protein is a nutritious protein source that is relatively free from allergens commonly associated with soy, milk and other grains. As such, rice proteins are valuable because they are hypoallergenic and can be used as a clean label modifier.

Rice protein is high in the sulfur-containing amino acids, cysteine and methionine, but low in lysine.

The rice protein may, for example, be an organic rice protein isolate.

### Enzymatic Hydrolysis

In certain embodiments, rice protein is subjected to enzymatic hydrolysis, wherein the rice protein is contacted with one or more enzyme(s) under conditions and for a period of time suitable for the enzyme(s) to at least partially break down the rice protein. All enzymes should be food grade.

In one embodiment, the enzyme(s) used for enzymatic hydrolysis may, for example, be selected from proteolytic enzymes. Proteolytic enzymes catalyse the hydrolysis of proteins and peptides. Proteolytic enzymes include, for example, proteinases, which hydrolyze proteins to form small peptides, and peptidases, which further hydrolyze small peptides to form amino acids. The proteolytic enzyme(s) may, for example, have endopeptidase activity (attack internal peptide bonds) and/or exopeptidase activity (attack peptide bonds at the end of the protein or peptide such as amino- or carboxypeptidases).

Proteolytic enzymes include, for example, protease, peptidase, glutaminase (e.g. L-glutamine-amido-hydrolase (EC 3.5.1.2)), endoprotease, serine endopeptidase, subtilisin peptidase (EC 3.4.21.62), serine protease, threonine protease, cysteine protease, aspartic acid protease, glutamic acid protease, trypsin, chymotrypsin (EC 3.4.21.1), pepsin, papain, and elastase.

Proteolytic enzymes (EC 3.4 and EC 3.5) are classified by an EC number (enzyme commission number), each class comprises various known enzymes of a certain reaction type. EC 3.4 comprises enzymes acting on peptide bonds (peptidases/proteinases) and EC 3.5 comprises enzymes that act on carbon-nitrogen bonds other than peptide bonds.

Examples for EC 3.4 include, for example, the following: aminopeptidase (EC 3.4.11), dipeptidase (3.4.13), dipeptidyl-peptidase (3.4.14), peptidyl-dipeptidase (3.4.15), serine-carboxypeptidase (3.4.16), metallocarboxypeptidase (3.4.17), cysteine-carboxypeptidase (3.4.18), omegapeptidase (3.4.19), serine-endopeptidase (3.4.21), cysteine-endopeptidase (3.4.22), aspartate-endopeptidase (3.4.23), metalloendopeptidase (3.4.24), threonine-endopeptidase (3.4.25).

Examples for EC 3.5 include, without limitation, proteolytic enzymes that cleave in linear amides (3.5.1), for example, without limitation, glutaminase (EC 3.5.1.2).

Various proteolytic enzymes, suitable for food-grade applications, are commercially available from suppliers such as Novozymes, Amano, Biocatalysts, Bio-Cat, Valey Research (now subsidiary of DSM). EDC (Enzyme Development Corporation), and others. Some examples include: Neutrase®, Alcalase®, Protamex®, and Flavorzyme®, (available from Novozymes); the Promod® series: e.g. 215P, 439L, 523MDP, 782MDP, 845MDP and 903MDP, Flavorpro®937MDP, 852MDP, 795MDP, 766MDP, 750MDP, P523MDP (available from Biocatalysts); Protin PC10, Umamizyme®, Peptidase R (or 723), protease A, protease M, protease N, protease P, and Thermoase GL30 (available from Amano); Validase® AFP and Validase® FPII (available from Valey Research); Fungal protease, Exo-protease, Papain, Bromelain, and the Enzeco® series of proteases and peptidases (available from EDC).

The enzymatic hydrolysis will be performed under conditions suitable for all the enzymes involved. As will be apparent to the skilled person, the temperature and pH should be within a suitable range for hydrolysis to occur to the desired degree. The incubation length will vary accordingly, with shorter incubations when conditions are nearer to the optimum conditions. Necessary ions, if required or beneficial for the chosen enzymes may be present. Subjecting the incubated mixture to agitation, for example by stirring (e.g. at 50 to 500 rpm or 100 to 200 rpm) may improve the hydrolysis.

The enzymatic hydrolysis may, for example, be performed at a temperature less than the temperature at which the enzymes denature. The temperature may, for example, be selected to give a desired reaction rate. The enzymatic hydrolysis may, for example, be performed at a temperature ranging from about 35° C. to about 80° C. For example, the enzymatic hydrolysis may be performed at a temperature ranging from about 40° C. to about 75° C. or from about 45° C. to about 70° C.

The enzymatic hydrolysis may, for example, be performed at a pH at which the enzymes do not denature. The pH may, for example, be selected to give a desired reaction rate. The enzymatic hydrolysis may, for example, be performed at a pH ranging from about 7 to about 8.5, for example from about 7.5 to about 8.5, for example from about 7.9 to about 8.3.

The enzymatic hydrolysis may, for example, take place for a period of time ranging from about 1 hour to about 48 hours. For example, the enzymatic hydrolysis may take place for a period of time ranging from about 2 hours to about 48 hours or from about 4 hours to about 36 hours or from about 6 hours to about 24 hours or from about 8 hours to about 16 hours.

### Further Processing Steps

The product of the enzymatic hydrolysis may, for example, be used directly as a taste modifying ingredient. However, the methods may, for example, comprise one or more additional steps. In one embodiment, the rice protein that is subjected to the enzymatic hydrolysis may, for example, be an aqueous slurry of rice protein. Thus, in certain embodiments, the method may comprise combining the rice protein with water and a buffer solution prior to the enzymatic hydrolysis. The aqueous slurry of rice protein may, for example, comprise at least about 5 wt % rice protein, for example at least about 10 wt % rice protein, for example at least about 15 wt % rice protein.

In one embodiment, the reaction mixture after incubation was cooled to room temperature and the mixture was submitted to a separation step, for example by centrifugation, so as to recover the supernatant. In accordance with the present disclosure, the supernatant can be either maintained as it is in liquid form or converted into a powder using mild conditions, for example, spray drying or freeze drying.

### Products

The taste modifying ingredient made by the enzymatic hydrolysis described herein may be used directly in flavour compositions and/or food compositions or may undergo further processing as described above. The taste modifying ingredient may, for example, be considered to be a natural product for fox labelling and/or food regulation reasons.

The final form of the taste modifying ingredient may be chosen according to methods well known in the art and will depend on the particular food application. For liquid foods, the taste modifying ingredient can be used without further processing in its liquid form. For dry applications, the spray-dried concentrated taste modifying ingredient can be used. The taste modifying ingredient may be directly added to food products, or may be provided as part of a flavour composition for flavouring or seasoning food products.

According to the present disclosure, flavour compositions may include a characterizing flavour and a taste modifying composition. The term “characterizing flavour” refers to a flavour that is perceived by an individual to be predominant upon consumption by the individual.

In one embodiment, the taste modifying compositions include the taste modifying ingredient derived from rice protein. The characterizing flavour and the taste modifying composition should be present in the flavour composition in an organoleptically effective amount. This amount will depend upon the nature of the characterizing flavour and taste modifying composition, as well as the nature of the flavour composition and the effect that is desired to be achieved, and it is within the purview of the skilled person to experiment with the desired amounts.

Flavour compositions may also contain one or more food grade excipient(s). Suitable excipients for flavour compositions are well known in the art and include, for example, without limitation, solvents (including water, alcohol, ethanol, oils, fats, vegetable oil, and miglyol), binders, diluents, disintegrating agents, lubricants, flavouring agents, colouring agents, preservatives, antioxidants, emulsifiers, stabilisers, flavour-enhancers, sweetening agents, anti-caking agents, and the like. Examples of such carriers or diluents for flavours may be found e.g. in “Perfume and Flavour Materials of Natural Origin”, S. Arctander. Ed., Elizabeth, N.J., 1960; in “Perfume and Flavor Chemicals”, S. Arctander, Ed., Vol. I & 11. Allured Publishing Corporation, Carol Stream, USA, 1994; in “Flavourings”, E. Ziegler and H. Ziegler (ed.), Wiley-VCH Weinheim, 1998, and “CTFA Cosmetic Ingredient Handbook”, J. M. Nikitakis (ed.), 1st ed., The Cosmetic, Toiletry and Fragrance Association, Inc., Washington, 1988.

The flavour composition may have any suitable form, for example liquid or solid, wet or dried, or in encapsulated form bound to or coated onto carriers/particles or as a powder. The flavour composition may include the characterizing flavour in an amount from about 0.01 to about 10%, in another embodiment from about 0.01 to about 5%, in yet another embodiment from about 0.01 to about 1%, or any individual number within the range, by weight of the flavour composition. In another embodiment, a consumable may include the characterizing flavour in an amount from about 0.001 to about 0.5%, in another embodiment from about 0.01 to about 0.3%, in yet another embodiment from about 0.02 to about 0.1%, or any individual number within the range, by weight of the consumable.

In a typical embodiment, the flavour composition includes from about 0.01% to about 10% of the taste modifying composition, by weight of the flavour composition, and depending upon the particular application desired. In one embodiment, the flavour composition comprises from about 0.01% to about 5% of the taste modifying composition, by weight of the flavour composition. In another embodiment, the flavour composition may comprise from about 0.01% to about 1% or any individual number within the range of the taste modifying composition, by weight of the flavour composition.

In another embodiment, a consumable may include the taste modifying composition in an amount from about 0.001 to about 1.0%, in another embodiment from about 0.01 to about 0.5%, in yet another embodiment from about 0.1 to about 0.2%, or any individual number within the range, by weight of the consumable.

In a typical embodiment, the food product may include from about 5 to about 50 ppm of the taste modifying ingredient, and depending upon the particular application desired. According to certain embodiments, the amount of taste modifying ingredient present in a consumable may be in the concentration of from at least about 5 ppm to about 35 ppm. According to certain embodiments, the amount of taste modifying ingredient present in a consumable may be in the concentration of from at least about 10 ppm to about 25 ppm.

The term “food product” is used in a broad meaning to include any product placed into the oral cavity but not necessarily ingested, including, for example, food, beverages, nutraceuticals and dental care products including mouth wash.

Food products include cereal products, rice products, pasta products, ravioli, tapioca products, sago products, baker's products, biscuit products, pastry products, bread products, confectionery products, dessert products, gums, chewing gums, chocolates, ices, honey products, treacle products, yeast products, salt and spice products, savoury food products, mustard products, vinegar products, sauces (condiments), processed foods, cooked fruits and vegetable products, meat and meat products, meat analogues/substitutes/alternatives, jellies, jams, fruit sauces, egg products, dairy products (including milk), cheese products, butter and butter alternative products, milk alternative products, soy products (e.g. soy “milk”), edible oils and fat products, medicaments, beverages, juices, fruit juices, vegetable juices, food extracts, plant extracts, meat extracts, condiments, nutraceuticals, gelatins, tablets, lozenges, drops, emulsions, elixirs, syrups, and combinations thereof.

Processed foods include margarine, peanut butter, soup (clear, canned, cream, instant, UHT), gravy, canned juices, canned vegetable juice, canned tomato juice, canned fruit juice, canned juice drinks, canned vegetables, pasta sauces, frozen entrees, frozen dinners, frozen hand-held entrees, dry packaged dinners (macaroni & cheese, dry dinners-add meat, dry salad/side dish mixes, dry dinners-with meat). Soups may be in different forms including condensed wet, ready-to-serve, ramen, dry, and bouillon, processed and pre-prepared low-sodium foods.

Of particular interest are, for example, dairy products such as milk (e.g. cow's milk, goat's milk, sheep's milk, camel's milk), cream, butter, cheese, yoghurt, ice cream, and custard. The dairy products may, for example, be sweetened or unsweetened. The dairy products (e.g. milk) may, for example, be full-fat, low-fat, or non-fat.

Dairy alternative products are also of particular interest. Dairy alternative products are plant-based products that do not encompass true dairy products that have been obtained from an animal. For example, dairy alternative products include alternative “milk”, “cream”, and “yoghurt” products which may, for example, be derived from soy, almond, rice, pea, coconut, and nuts (e.g. cashew). The dairy alternative products may, for example, be sweetened or unsweetened.

Of further particular interest are, for example, beverages including beverage mixes and concentrates, including, for example, alcoholic and non-alcoholic ready to drink and dry powdered beverages, carbonated and non-carbonated beverages, e.g., sodas, fruit or vegetable juices, alcoholic and non-alcoholic beverages. The beverages may, for example, be sweetened or unsweetened.

Of further particular interest are, for example, food products traditionally high in sodium salt with a reduced sodium salt concentration, including condiments and sauces (cold, warm, instant, preserved, sate, tomato, BBQ Sauce, Ketchup, mayonnaise and analogues, bechamel), gravy, chutney, salad dressings (shelf stable, refrigerated), batter mixes, vinegar, pizza, pasta, instant noodles, french fries, croutons, salty snacks (potato chips, crisps, nuts, tortilla-tostada, pretzels, cheese snacks, corn snacks, potato-snacks, ready-to-eat popcorn, microwaveable popcorn, caramel corn, pork rinds, nuts), crackers (Saltines, ‘Ritz’ type), “sandwich-type” cracker snacks, breakfast cereals, cheeses and cheese products including cheese analogues (reduced sodium cheese, pasteurized processed cheese (food, snacks & spreads), savoury spreads, cold pack cheese products, cheese sauce products, meats, aspic, cured meats (ham, bacon), luncheon/breakfast meats (hotdogs, cold cuts, sausage), soya-based products, tomato products, potato products, dry spice or seasoning compositions, liquid spice or seasoning compositions including pesto, marinades, and soup-type/meal-alternative beverages, and vegetable juices including tomato juice, carrot juice, mixed vegetable juices and other vegetable juices.

The flavour compositions and food products may, for example, comprise one or more sweeteners. Examples of sweeteners that may be used in the sweetened compositions are disclosed, for example, in WO 2016/038617, the contents of which are incorporated herein by reference.

The one or more sweeteners may comprise one or more natural sweeteners and/or one or more artificial sweeteners. The one or more sweeteners may, for example, be selected from sucrose, fructose, glucose, xylose, arabinose, rhamnose, tagatose, allulose, trehalose, isomaltulose, steviol glycosides (e.g, rebaudioside A, rebaudioside B, rebaudioside C, rebaudioside D, rebaudioside E, rebaudioside F, rebaudioside G, rebaudioside H, rebaudioside I, rebaudioside J, rebaudioside K, rebaudioside L, rebaudioside M, rebaudioside N, rebaudioside O, dulcoside A, dulcoside B, rubusoside, naringin dihydrochalcone, stevioside), mogrosides (e.g. grosvenorine II, grosvenorine I, 11-O-mogroside II (I), 11-O-mogroside II (II), 11-O-mogroside II (III), mogroside II (I), mogroside II (II), mogroside II (III), 11-dehydroxy-mogroside III, 11-O-mogroside III, mogroside III (I), mogroside III (II), mogroside IIIe, mogroside IIIx, mogroside IV (I) (siamenoside), mogroside IV (II), mogroside IV (III), mogroside IV (IV), deoxymogroside V (I), deoxymogroside V (II), 11-O-mogroside V (I), mogroside V isomer, mogroside V, iso-mogroside V, 7-O-mogroside V, 11-O-mogroside VI, mogroside VI (I), mogroside VI (II), mogroside VI (III) (neomogroside) and mogroside VI (IV)), stevia, trilobatin, rebusoside, aspartame, advantame, agave syrup, acesulfame potassium (AceK), high fructose corn syrup, neotame, saccharin, sucralose, high fructose corn syrup, starch syrup, Luo Han Guo extract, neohespiridin, dihydrochalcone, naringin, sugar alcohols (e.g. sorbitol, xylitol, inositol, mannitol, erythritol), cellobiose, psicose, and cyclamate.

### Uses

The taste modifying ingredient obtained by and/or obtainable by the methods described herein may, for example, be added to food products (e.g. as part of a flavour composition) to modify/enhance the flavour or sweetness of the food product.

The taste modifying ingredient obtained by and/or obtainable by the methods described herein may, for example, be used to improve the mouthfeel of a food product and/or to mask off-notes of a food product and/or to improve the sweetness of a food product and/or to enhance the saltiness of a food product and/or to act as a prebiotic in a food product.

Thus, there is also provided herein a method of providing a food product having improved mouthfeel and/or reduced off-notes and/or improved sweetness and/or enhanced saltiness and/or use as a prebiotic, the method comprising admixing the taste modifying ingredient obtained by and/or obtainable by the methods described herein with the food product.

In general terms, “mouthfeel” refers to the complexity of perceptions experienced in the mouth as influenced by the aroma, taste, and texture qualities of food and beverage products. From a technical perspective, however, mouthfeel sensations are specifically associated with physical (e.g. tactile, temperature) and/or chemical (e.g. pain) characteristics perceived in the mouth via the trigeminal nerve. Accordingly, they are a consequence of oral-tactile stimulations and involve mechanical, pain and temperature receptors located in the oral mucosa, lips, tongue, cheeks, palate and throat.

Mouthfeel perceptions include, for example, one or more of texture—astringent, burning, cold, tingling, thick, biting, fatty, oily, slimy, foamy, melting, sandy, chalky, watery, acidic, lingering, metallic, body, body sweet, carbonation, cooling, warming, hot, juicy, mouth drying, numbing, pungent, salivating, spongy, sticky, fullness, cohesiveness, density, fracturability, graininess, grittiness, gumminess, hardness, heaviness, moisture absorption, moisture release, mouthwatering, mouthcoating, roughness, slipperiness, smoothness, uniformity, uniformity of bite, uniformity of chew, viscosity, fast-diffusion, full body, salivation and retention.

By “improvement of mouthfeel” it is meant that any one or more of desired mouthfeel perceptions is/are enhanced and/or that any one or more undesirable mouthfeel perceptions is/are reduced. In particular, one or more of the following perceptions may be enhanced by the product and methods described herein: creamy sour, acidic dairy, sweet, salty, umami, By “masking of off-notes” it is meant that the intensity and/or length of perception of undesirable attributes in a food product is reduced, as analysed by trained panelists when comparing food comprising an ingredient with off-note masking to food without an added off-note masking ingredient.

By “improvement in sweetness” it is meant the effect of the taste modifying ingredient on the sweetness characteristics of a food which are found to be more favourable as analysed by trained panelists when comparing food comprising an ingredient with sweetness improving effect to food without an added sweetness improving ingredient. The improvement in sweetness may, for example, provide sweetness characteristics that are more similar to the sweetness characteristics of sucrose.

In certain embodiments, the taste modifying ingredient may be used to inhibit the degradation of sweeteners and/or sweetness enhancers and to strengthen the sweetness impact of the food product (e.g. sweetened food product). The sweetness impact relates to the length of time it takes before the sweetness is initially detected and the intensity at which the sweetness is initially detected. The taste modifying ingredient may, for example, decrease the amount of time before the sweetness is initially detected and/or increase the intensity at which the sweetness is initially detected.

The degree of sweetness and other sweetness characteristics described herein may be evaluated by a tasting panel of flavorists, for example as described in the examples below.

According to other embodiments, the disclosed method may be used to reduce or eliminate off-notes imparted by non-animal derived protein such as plant protein. Exemplary plant proteins include soy protein and pea protein. As used herein, soy includes all consumables containing soy in any form, including soybean oil used either alone, in combination, for example as a nutraceutical, or as a medicament, soy bean curd, soy milk, soy butter or soy paste. The plant protein may comprise algae (such as spirulina), beans (such as black beans, canelli beans, kidney beans, lentil beans, lima beans, pinto beans, soy beans, white beans), broccoli, edamame, nuts (such as almonds, brazil nuts, cashews, peanuts, pecans, hazelnuts, pine nuts, walnuts), peas (such as black eyed peas, chickpeas, green peas), potatoes, oatmeal, seeds (such as chia, flax, hemp, pumpkin, sesame, sunflower), grain (rice, millet, maize, barley, wheat, oat, sorghum, rye, teff, triticale, amaranth, buckwheat, quinoa), seitan (i.e., wheat gluten-based), tempeh, tofu, mycoprotein or fungal protein; insects and leaf protein and mixtures thereof.

In one example, the taste modifying ingredient derived from rice protein is selected based on its ability to block, mask or modify the undesirable off-note(s) in a particular non-animal protein. Various non-animal proteins provide undesirable off-notes. Particularly, undesirable off-notes are the beany, bitter, grassy, astringent, earthy, chalky, and rancid off-notes. The term off-note refers to an unpleasant after taste that develops over time after consumption of consumables. The addition of the taste modifying ingredient derived from rice protein will block, mask or modify the off-notes and make them less apparent or unnoticeable. Non-animal proteins will thereby lose their beany/bitter/grassy/astringent/earthy/chalky/rancid taste.

In another embodiment, it was surprisingly and unexpectedly found that 1,3-propanediol may be used in combination with the taste modifying ingredient derived from rice protein in consumables to reduce or eliminate off-notes imparted by non-animal derived protein. 1,3-propanediol is a polar compound that can be prepared from corn sugar. Generally, 1,3-propanediol is included in consumables in an amount such that 1,3-propanediol does not itself provide flavor to the food or beverage and is not perceived through taste as being included in the product. For example, 1,3-propanediol is included in an amount generally considered to be below the organoleptically perceptible flavor threshold for the average consumer. In other words, a comparative product containing no 1,3-propanediol is not perceptibly different in taste than a product containing 1,3-propanediol. 1,3-propanediol is commercially sold as ZEMEA® from DuPont Tate & Lyle BioProducts (Wilmington, Del.) but other sources of 1,3-propanediol may also be used.

According to other embodiments, the disclosed method may be used to reduce or eliminate off-notes imparted by meat analog products containing non-animal protein. “Meat analog” is a food product that approximates the aesthetic qualities and/or chemical characteristics of certain types of meat. The term Meat analogue includes those prepared with textured vegetable proteins (TVP), high moisture meat analogue (HMMA) and low moisture meat analogue (LMMA) products. In another embodiment, it was surprisingly and unexpectedly found that 1,3-propanediol may be used in combination with the taste modifying ingredient derived from rice protein in meat analog products containing non-animal protein to reduce or eliminate off-notes.

According to certain embodiments, the amount of 1,3-propandiol present in a meat analog product or other non-animal protein containing consumable may be in the concentration of from at least about 50 ppm to about 1000 ppm. According to certain embodiments, the amount of 1,3-propandiol may be in the concentration of from at least about 100 ppm to about 500 ppm. According to certain embodiments, the amount of 1,3-propandiol may be in the concentration of from at least about 150 ppm to about 300 ppm. According to certain embodiments, the amount of 1,3-propandiol may be in the concentration of about 200 ppm.

### Meat Analog Composition and Extrusion Process

Food scientists have devoted much time developing methods for preparing acceptable meat-like food applications, such as beef, pork, poultry, fish, and shellfish analogs, from a wide variety of non-animal proteins. One such approach is texturization into fibrous meat analogs, for example, through extrusion processing. The resulting meat analog products exhibit improved meat-like visual appearance and improved texture.

Meat analog products are produced with high moisture content and provide a product that simulates the fibrous structure of animal meat and has a desirable meat-like moisture, texture, mouthfeel, flavor and color.

Texturization of protein is the development of a texture or a structure via a process involving heat, and/or shear and the addition of water. The texture or structure will be formed by protein fibers that will provide a meat-like appearance and perception when consumed. The mechanism of texturization of proteins starts with the hydration and unfolding of a given protein by breaking intramolecular binding forces by heat and/or shear. The unfolded protein molecules are aligned and bound by shear, forming the characteristic fibers of a meat-like product. In one embodiment, polar side chains from amino acids form bonds with linear protein molecules and the bonds will align protein molecules, forming the characteristic fibers of a meat-like product.

To make non-animal proteins palatable, texturization into fibrous meat analogs, for example, through extrusion processing has been an accepted approach. Due to its versatility, high productivity, energy efficiency and low cost, extrusion processing is widely used in the modern food industry. Extrusion processing is a multi-step and multifunctional operation, which leads to mixing, hydration, shear, homogenization, compression, deaeration, pasteurization or sterilization, stream alignment, shaping, expansion and/or fiber formation. Ultimately, the non-animal protein, typically introduced to the extruder in the form of a dry blend, is processed to form a fibrous material.

More recent developments in extrusion technology have focused on using twin screw extruders under high moisture (40-80%) conditions for texturizing non-animal proteins into fibrous meat alternatives in the high moisture twin screw process, also known as “wet extrusion”, the raw materials, predominantly non-animal proteins such as soy and/or pea protein, are mixed and fed into a twin-screw extruder, where a proper amount of water is dosed in and all ingredients are further blended and then melted by the thermo-mechanical action of the screws. The realignment of large protein molecules, the laminar flow, and the strong tendency of stratification within the extruder's long slit cooling die contribute to the formation of a fibrous structure. The resulting wet-extruded products tend to exhibit improved whole muscle meat-like visual appearance and improved palatability. Therefore, this extrusion technology shows promise for texturizing non-animal proteins to meet increasing consumer demands for healthy and tasty foods.

Texturization processes may also include spinning, simple shear flow, and simple shear flow and heat in a Couette Cell (“Couette Cell” technology). The spinning process consists of unfolding protein molecules in a high alkaline pH solution, and coagulating the unfolded protein molecules by spraying the protein alkaline solution into an acid bath. The spraying is made by a plate with numerous fine orifices. The protein coagulates forming fibers as soon as it gets in contact with the acid medium. The fibers are then washed to remove remaining acid and/or salts formed in the process. A Couette Cell is a cylinder-based device where the inner cylinder rotates and the outer cylinder is stationary, being easy to scale up. The Couette Cell operates under the same principle of forming protein fibers by subjecting the protein to heat and shear in the space between the stationary cylinder and the rotational cylinder.

With respect to simple shear flow and heat in a Couette Cell, this process can induce fibrous structural patterns to a granular mixture of non-animal proteins at mild process conditions. This process is described in “On the use of the Couette Cell technology for large scale production of textured soy-based meat replacers”, Journal of Food Engineering 169 (2016) 205-213, which is incorporated herein by reference.

Meat analog products having qualities (for example, texture, moisture, mouthfeel, flavor, and color) similar to that of whole muscle animal meat may be produced using non-animal proteins formed using extrusion under conditions of relatively high moisture. In one embodiment, meat analog products may include non-animal protein, one or more of flour, starch, and edible fiber, an edible lipid material.

In certain compositions, the amount of non-animal protein included in the mixture to be extruded includes no more than about 90% by weight of the dry ingredients. For example, the amount of non-animal protein present in the ingredients utilized to make meat analog products according to the present disclosure may range from about 3% to about 90% by weight of the dry ingredients. In another embodiment, the amount of non-animal protein present in the ingredients utilized to make meat analog products according to the present disclosure may range from about 10% to about 80% by weight of the dry ingredients. In a further embodiment, the amount of non-animal protein present in the dry ingredients utilized to make meat analog products according to the present disclosure may range from about 25% to about 50% by weight. In another further embodiment, the amount of non-animal protein present in the dry ingredients utilized to make meat analog products according to the present disclosure may be about 40%.

The term “dry ingredients” includes all the ingredients in the mixture to be extruded except for added water and ingredients added with the added water (i.e., the “wet ingredients”).

In one embodiment, the non-animal protein ingredients are isolated from soybeans. Suitable soybean derived protein-containing ingredients include soy protein isolate, soy protein concentrate, soy flour, and mixtures thereof. The soy protein materials may be derived from whole soybeans in accordance with methods generally known in the art. In another exemplary embodiment, the non-animal protein ingredients are isolated from grain, legume or pulses, seed and oilseed, nut, algal, mycoprotein or fungal protein, insects, leaf protein and combinations thereof as described herein.

In addition to the foregoing, the meat analog product includes water at a relatively high amount. In one embodiment, the total moisture level of the mixture extruded to make the meat analog product is controlled such that the meat analog product has a moisture content that is at least about 50% by weight. To achieve such a high moisture content, water is typically added to the ingredients. Although, a relatively high moisture content is desirable, it may not be desirable for the meat analog product to have a moisture content much greater than about 65%. As such, in one embodiment the amount of water added to the ingredients and the extrusion process parameters are controlled such that the meat analog product (following extrusion) has a moisture content that is from about 40% to about 65% by weight.

Among the suitable extrusion apparatuses useful in the practice of the described process is a commercially available double barrel, twin-screw extruder apparatus such as a Wenger TX 52 model manufactured by Wenger (Sabetha, Kans.).

The screws of a twin-screw extruder can rotate within the barrel in the same or opposite directions. Rotation of the screws in the same direction is referred to as single flow or co-rotating whereas rotation of the screws in opposite directions is referred to as double flow or counter-rotating. The speed of the screw or screws of the extruder may vary depending on the particular apparatus; however, it is typically from about 100 to about 450 revolutions per minute (rpm). Generally, as the screw speed increases, the density of the extrudate will decrease. The extrusion apparatus contains screws assembled from shafts and worm segments, as well as mixing lobe and ring-type shearing elements as recommended by the extrusion apparatus manufacturer for extruding non-animal protein material.

The extrusion apparatus generally comprises a plurality of heating zones through which the protein mixture is conveyed under mechanical pressure prior to exiting the extrusion apparatus through an extrusion die. The temperature in each successive heating zone generally exceeds the temperature of the previous heating zone by between about 10° C. to about 70° C. In one embodiment, the dry premix is transferred through multiple heating zones within the extrusion apparatus, with the protein mixture heated to a temperature of from about 25° C. to about 170° C. such that the molten extrusion mass enters the extrusion die at a temperature of from about 170° C. In one embodiment, the protein mixture is heated in the respective heating zones to temperatures of about 25° C., about 40° C., about 95° C., about 150° C. and about 170° C.

The pressure within the extruder barrel is typically between about 30 psig and about 500 psig, or more specifically between about 50 psig and about 300 psig. Generally, the pressure within the last two heating zones is between about 50 psig and about 500 psig, even more specifically between about 50 psig to about 300 psig. The barrel pressure is dependent on numerous factors including, for example, the extruder screw speed, feed rate of the mixture to the barrel, feed rate of water to the barrel, and the viscosity of the molten mass within the barrel.

Water along with additional “wet ingredients” are injected into the extruder barrel to hydrate the non-animal protein mixture and promote texturization of the proteins. As an aid in forming the molten extrusion mass, the water may act as a plasticizing agent. Water may be introduced to the extruder barrel via one or more injection jets. The rate of introduction of water to the barrel is generally controlled to promote production of an extrudate having the aforementioned desired characteristics, such as an extrudate with a moisture content as described above.

### Textured Vegetable Proteins (TVP)/Low Moisture Meat Analogue (LMMA)

Textured vegetable proteins (TVPs) can be defined as food products made from edible protein sources and characterised by having structural integrity and identifiable texture such that each unit will withstand hydration in cooking and other procedures used in preparing the food for consumption. A majority of TVPs produced today are produced by extrusion technology. These TVPs are often rehydrated with 60-65% moisture and blended with other ingredients including, but not limited to, binders, meats, other TVPs, flavours, excipient, fats, oils, or seasonings.

The low-moisture meat analog (LMMA) product is most often cut with an extruder knife at the extruder die to form the finished product size and shape. Drying after extrusion, to remove moisture, improves storage, handling, and shelf-stability. These LMMAs are often rehydrated with 60-70% moisture. Additionally, other food ingredient items can be added to improve finished product functionality and appearance, including, but not limited to, oil, other proteins, salt, seasonings, flavours, maskers, enhancers, or binders. Generally re-hydrated LMMA contains 40-80% moisture, 0-5% oil, 25-60% protein.

A typical formulation of LMMA contains water, soy concentrates, soy isolates, oil, a binder (e.g. cellulose, vital wheat gluten) and flavours, maskers, seasonings, etc. that provide a taste and texture closer to an animal meat product.

### EXAMPLES

The following examples are given solely for the purpose of illustration and are not to be construed as limitations of the present invention, as many variations of the invention are possible without departing from the spirit and scope of the present disclosure.

### Enzymatic Hydrolysis of Rice Protein (TMI-T)

Taste modifying ingredient was made by mixing 12.3 g of organic rice protein isolate with 87.7 g of water in a clean, sanitized tank. 0.6-1.1% NaOH (50% solution) was added to the mixture to make a mixture between pH 7.9 and 8.3. At room temperature, 60 mg of a proteolytic enzyme was added to the slurry and incubated at 70° C. for 1 hour and 40 minutes with continuous agitation. The mixture was then terminated at 95° C. for 45 minutes. The mixture was cooled down to room temperature, centrifuged and the supernatant was recovered as TMI-T. Water was then removed by freeze drying.

Taste modifying ingredient (TMI-T) was then tested in a neutral carbonated soft drink beverage. Beverages were prepared from the ingredients listed below in Tables 1 and 2:

TABLE 1
Neutral CSD Base for Full Sugar and Reduced Sugar Beverages

| Ingredients | % Weight (g) |
|---|---|
| Sugar Syrup - 65°Brix | 80.00 |
| Sodium Benzoate | 0.15 |
| Citric Acid | 0.55 |
| Water | 19.30 |
| Concentrated Syrup Base | 100.00 |

TABLE 2
Neutral Carbonated Soft Drink Beverage

| | | Red. Sugar | Red. Sugar | Red. Sugar | Red. Sugar |
|---|---|---|---|---|---|
| | Full Sugar | w/FMP | w/FMP | w/Reb M | w/Reb M |
| Ingredients (g) | (Control) | Ex. 1 | Ex. 2 | Ex. 3 | Ex. 4 |
| Concentrated Syrup Base | 40.00 | 28.00 | 28.00 | 28.00 | 28.00 |
| from Table 1 | | | | | |
| Sweetness Flavour | | 0.20 | 0.20 | | |
| w/stevia FMP* | | | | | |
| Reb M | | | | 0.01 | 0.01 |
| TMI-T Sol. 5% PG/WA | | | 0.20 (50 ppm) | | 0.20 (50 ppm) |
| (75%/25%) | | | | | |
| Carbonated Water, 3.7 | qs p/200 ml | qs p/200 ml | qs p/200 ml | qs p/200 ml | qs p/200 ml |
| vol: | | | | | |
| Total Carbonated | 200.00 | 200.00 | 200.00 | 200.00 | 200.00 |
| Beverage | | | | | |
| Brix | 10.40° | 7.28° | 7.28° | 7.28° | 7.28° |
| pH | 3.14 | 3.29 | 3.30 | 3.30 | 3.26 |

*Flavour with Modifying Properties (FMP)

Beverages according to the present disclosure were prepared as follows:
Prepare one large batch of Concentrated Syrup Base for Full Sugar, enough to cover reference bottles.
Prepare one large batch of Concentrated Syrup Base Reduced 301% Sugar, enough to cover all testing pointed at 7.28° Brix.
In appropriate 200 ml glass bottle, the Concentrated Syrup Base and other ingredients were combined.

Prepare 8 bottles of each Example were filled with still drinkable water

4 bottles of each Example are stored in refrigerator (4-6° C.).
4 bottles of each trial are stored into chamber-hot under 36-37° C., accelerated conditions.

### Stability Results

The beverages from Table 2 (Control 2 Examples 14) were tested at time zero and after 2, 4 and 8 weeks comparing a refrigerated sample against chamber/hot sample. The results are shown in Table 3. Organoleptic evaluation was undertaken for each Example and was carried out by flavorists (pass/fail+descriptive analysis).

TABLE 3
Stability Study Results

| | Initial | 2 weeks | 4 weeks | 6 weeks |
|---|---|---|---|---|
| Examples | | | | |
| (Refrigerated) | | | | |
| Control | Pass | Pass | Pass | Pass |
| Ex. 1 | Pass | Pass | Pass | Accepted |
| Ex. 2 | Pass | Pass | Pass | Pass |
| Ex. 3 | Pass | Pass | Pass | Pass |
| Ex. 4 | Pass | Pass | Pass | Pass |
| Examples | | | | |
| (Chamber--Hot) | | | | |
| Control | N/A | Pass | Pass | Accepted |
| Ex. 1 | N/A | Accepted | Fail | Fail |
| Ex. 2 | N/A | Pass | Pass | Pass |
| Ex. 3 | N/A | Pass | Fail | Fail |
| Ex. 4 | N/A | Pass | Pass | Pass |

The beverage compositions of Table 3 (aged 6 weeks, chamber-hot) were taste tested by 5 flavorists and they were asked to describe the beverages.

TABLE 4

| Sample | Taste Results |
|---|---|
| Full sugar reference (Control) | Sweet intensity, sugary, juiciness all ok, body |
| | full, slight caramelic/cooked |
| Reduced sugar w/stevia FMP (Ex. 1) | Lower sweet intensity, increased leaves and |
| | hay-like note, slightly higher astringency |
| Reduced sugar w/stevia FMP + TMI-T (Ex. 2) | Sweetness, body ok, slightly hay-like note, no |
| | astringency |
| Reduced sugar w/Reb M (Ex. 3) | Lower sweet intensity, slight body, slightly |
| | sugary, slightly caramelic, increased hay-like |
| | note, increased astringency |
| Reduced sugar w/Reb M + TMI-T (Ex. 4) | Sweet intensity, body, sugary all ok, reduced |
| | astringency, clean aftertaste |

From Tables 3 and 4 above, it can be seen that the inclusion of TMI-T improves the sweet intensity and sweetness overall quality of beverages including stevia FMP's or Reb M during shelf-life of carbonated soft drinks.

Taste modifying ingredient (TMI-T) was then tested in a neutral carbonated soft drink beverage. Beverages were prepared from the ingredients listed below in Tables 1 and 2:

TABLE 5
Neutral Base for Full Sugar and Reduced Sugar Beverages

| Ingredients | % Weight (g) |
|---|---|
| Sugar Syrup - 65°Brix | 31.000 |
| Sodium Benzoate | 0.060 |
| Ascorbic Acid | 0.020 |
| Caramel Color | 0.036 |
| Citric Acid | 0.183 |
| Water | 8.701 |
| Concentrated Syrup Base | 40.000 |

TABLE 6
Non-Flavoured Still Beverage

| | | Red. Sugar | Red. Sugar | Red. Sugar | Red. Sugar |
|---|---|---|---|---|---|
| | Full Sugar | w/Reb M | w/Reb M | w/Reb A | w/Reb A |
| Ingredients (g) | (Control) | Ex. 5 | Ex. 6 | Ex. 7 | Ex. 8 |
| Concentrated Syrup Base | 40.00 | 21.60 | 21.60 | 21.60 | 21.60 |
| from Table 1 | | | | | |
| Reb A | | | | 0.10 | 0.10 |
| Reb M | | 0.10 | 0.10 | 0.01 | 0.01 |
| TMI-T Sol. 5% PG/WA | | | 0.10 (50 ppm) | | 0.10 (50 ppm) |
| (75%/25%) | | | | | |
| Still Water | qs p/200 ml | qs p/200 ml | qs p/200 ml | qs p/200 ml | qs p/200 ml |
| Total Still Beverage | 200.00 | 200.00 | 200.00 | 200.00 | 200.00 |
| Brix | 10.075° | 7.02° | 7.02° | 7.02° | 7.02° |

*Flavour with Modifying Properties (FMP)

Beverages according to the present disclosure were prepared as follows:
Prepare one large batch of Concentrated Syrup Base for Full Sugar, enough to cover reference bottles.
Prepare one large batch of Concentrated Syrup Base Reduced 30% Sugar, enough to cover all testing pointed at 7.02° Brix.
In appropriate 200 ml glass bottle, the Concentrated Syrup Base and other ingredients were combined.

Prepare 6 bottles of each Example were filled with still drinkable water.

3 bottles of each Example are stored in refrigerator (4-6° C.).
3 bottles of each trial are stored into chamber-hot under 36-37° C., accelerated conditions.

### Stability Results

The beverages from Table 6 (Control+Examples 5-8) were tested at time zero and after 4 and 8 weeks, comparing a refrigerated sample against chamber/hot sample. The results are shown in Table 7. Organoleptic evaluation was undertaken for each Example and was carried out by flavorists (pass/fail+descriptive analysis)

TABLE 7
Stability Study Results

| | Initial | 4 weeks | 8 weeks |
|---|---|---|---|
| Examples | | | |
| (Refrigerated) | | | |
| Control | Pass | Pass | Pass |
| Ex. 5 | Pass | Pass | Pass |
| Ex. 6 | Pass | Pass | Pass |
| Ex. 7 | Pass | Pass | Pass |
| Ex. 8 | Pass | Pass | Pass |
| Examples | | | |
| (Chamber--Hot) | | | |
| Control | N/A | Pass | Accepted |
| Ex. 5 | N/A | Fail | Fail |
| Ex. 6 | N/A | Pass | Pass |
| Ex. 7 | N/A | Fail | Fail |
| Ex. 8 | N/A | Pass | Pass |

The beverage compositions of Table 7 (aged 8 weeks, chamber-hot) were taste tested by flavorists and they were asked to describe the beverages.

TABLE 8

| Sample | Taste Results |
|---|---|
| Full sugar reference (Control) | Similar sweet upfront and intensity, slight |
| | sweeter/caramelic linger, high sugary and |
| | juicy, acidity increased, no astringency, |
| | slightly bitter, overall sweetness poor |
| Reduced sugar w/Reb M (Ex. 5) | Lower upfront, slightly reduced intensity, |
| | linger higher, reduced sugary, juicy and lower |
| | body, slightly bitter and increased astringency, |
| | overall sweetness reduced |
| Reduced sugar w/Reb M + TMI-T (Ex. 6) | Upfront improved, similar intensity, slightly |
| | reduced linger perceived, higher sugary, juicy |
| | and body, no bitterness, overall sweetness |
| | fresher, improved |
| Reduced sugar w/Reb A (Ex. 7) | Lower upfront, reduced intensity, higher sweet |
| | linger, reduced sugary, juiciness, bitter and |
| | astringency increased, overall sweetness poor |
| Reduced sugar w/Reb A + TMI-T (Ex. 8) | Improved upfront, slightly more intensity, |
| | slightly lower linger, increased sugary, juicy, |
| | no bitterness, no astringency, overall sweetness |
| | improved |

From Tables 7 and 8 above, it can be seen that the inclusion of TMI-T improves the sweet intensity and sweetness overall quality of beverages including Reb A or Reb M during shelf-life of still beverages.

### Pea Protein Beverages

Taste modifying ingredients were then tested in pea protein beverages. For purposes of the following examples, the taste modifying ingredients were prepared using varying percentages of Bromelein as the proteolytic enzyme and varying incubation times.

### Example 9

A pea protein beverage is prepared by mixing 3% pea protein isolate (Pisane® C9), 3% sucrose, 0.05% stabilizer (Gellan gum), and 0.4% natural vanilla flavor—dry weight in water.

### Example 10

A pea protein beverage was prepared in accordance with Example 9. 10 ppm of TMI (1% Bromelein, 3 hrs) was added to the pea protein beverage.

### Example 11

A pea protein beverage was prepared in accordance with Example 9. 10 ppm of TMI (2% Bromelein, 1.5 hrs) was added to the pea protein beverage.

### Example 12

A pea protein beverage was prepared in accordance with Example 9. 10 ppm of TMI (2% Bromelein, 3 hrs) was added to the pea protein beverage.

The pea protein beverage compositions of Examples 9-12 were were taste tested by 5 flavorists and they were asked to describe the beverages.

TABLE 9

| Sample | Taste Results |
|---|---|
| Example 9 (Control) | Vanilla, sweet, beany, astringent |
| Example 10 | Similar to control |
| Example 11 | Slightly less astringent, more sweet, less bitter |
| Example 12 | Less astringent, less bitter |

From Table 9 above, it can be seen that the inclusion of TMI results in a perceived reduction in off-notes from pea protein particularly in bitterness and astringency.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method for making a taste modifying ingredient, the method comprising the steps of:
a. subjecting a rice protein to enzymatic hydrolysis to obtain a reaction mixture;
b. separating the reaction mixture to obtain a supernatant; and
c. recovering the supernatant of the reaction mixture.

2. The method according to claim 1, wherein the rice protein is a rice protein isolate.

3. The method according to claim 1, wherein the rice protein is an aqueous slurry of rice protein.

4. The method according to claim 1, wherein the enzymatic hydrolysis uses one or more proteolytic enzymes.

5. The method according to claim 1, wherein the enzymatic hydrolysis is performed at a temperature ranging from about 35° C. to about 80° C.

6. The method according to claim 1, wherein the enzymatic hydrolysis takes place for a period of time ranging from about 1 hour to about 48 hours.

7. A flavour composition comprising:
a characterizing flavour; and
a taste modifying composition comprising a rice protein isolate.

8. The flavour composition according to claim 7, further comprising one or more sweeteners.

9. The flavour composition according to claim 8, wherein the one or more sweeteners are selected from sucrose, fructose, glucose, xylose, arabinose, rhamnose, tagatose, allulose, trehalose, isomaltulose, steviol glycosides, mogrosides, stevia, trilobatin, rubusoside, aspartame, advantame, agave syrup, acesulfame potassium (AceK), high fructose corn syrup, neotame, saccharin, sucralose, high fructose corn syrup, starch syrup, Luo Han Guo extract, neohespiridin, dihydrochalcone, naringin, sugar alcohols cellobiose, psicose, and cyclamate.

10. The flavour composition according to claim 9, wherein the steviol glycosides are selected from rebaudioside A, rebaudioside B, rebaudioside C, rebaudioside D, rebaudioside E, rebaudioside F, rebaudioside G, rebaudioside H, rebaudioside I, rebaudioside J, rebaudioside K, rebaudioside L, rebaudioside M, rebaudioside N, rebaudioside O, dulcoside A, dulcoside B, rubusoside, naringin dihydrochalcone, stevioside, and mixtures thereof.

11. The flavour composition according to claim 9, wherein the mogrosides are selected from grosvenorine II, grosvenorine I, 11-O-mogroside II (I), 11-O-mogroside II (II), 11-O-mogroside II (III), mogroside II (I), mogroside II (II), mogroside II (III), 11-dehydroxy-mogroside III, 11-O-mogroside III, mogroside III (I), mogroside III (II), mogroside IIIe, mogroside IIIx, mogroside IV (I) (siamenoside), mogroside IV (II), mogroside IV (III), mogroside IV (IV), deoxymogroside V (I), deoxymogroside V (II), 11-O-mogroside V (I), mogroside V isomer, mogroside V, iso-mogroside V, 7-O-mogroside V, 11-O-mogroside VI, mogroside VI (I), mogroside VI (II), mogroside VI (III) (neomogroside) and mogroside VI (IV), and mixtures thereof.

12. The flavour composition according to claim 9, wherein the sugar alcohols are selected from sorbitol, xylitol, inositol, mannitol, erythritol, and mixtures thereof.

13. The flavour composition according to claim 7, wherein the composition comprises from about 0.01% to about 1% by weight of the characterizing flavour.

14. The composition according to claim 7, wherein the flavour composition is in the form of an emulsion, a solution or a powder.

15. A beverage comprising:
a flavor composition comprising a characterizing flavour and a taste modifying composition; and
one or more sweeteners;
wherein the taste modifying composition comprises a rice protein isolate.

16. The beverage according to claim 15, wherein the one or more sweeteners are selected from sucrose, fructose, glucose, xylose, arabinose, rhamnose, tagatose, allulose, trehalose, isomaltulose, steviol glycosides, mogrosides, stevia, trilobatin, rubusoside, aspartame, advantame, agave syrup, acesulfame potassium (AceK), high fructose corn syrup, neotame, saccharin, sucralose, high fructose corn syrup, starch syrup, Luo Han Guo extract, neohespiridin, dihydrochalcone, naringin, sugar alcohols cellobiose, psicose, and cyclamate.

17. The beverage according to claim 16, wherein the steviol glycosides are selected from rebaudioside A, rebaudioside B, rebaudioside C, rebaudioside D, rebaudioside E, rebaudioside F, rebaudioside G, rebaudioside H, rebaudioside I, rebaudioside J, rebaudioside K, rebaudioside L, rebaudioside M, rebaudioside N, rebaudioside O, dulcoside A, dulcoside B, rubusoside, naringin dihydrochalcone, stevioside, and mixtures thereof.

18. The beverage according to claim 16, wherein the mogrosides are selected from grosvenorine II, grosvenorine I, 11-O-mogroside II (I), 11-O-mogroside II (II), 11-O-mogroside II (III), mogroside II (I), mogroside II (II), mogroside II (III), 11-dehydroxy-mogroside III, 11-O-mogroside III, mogroside III (I), mogroside III (II), mogroside IIIe, mogroside IIIx, mogroside IV (I) (siamenoside), mogroside IV (II), mogroside IV (III), mogroside IV (IV), deoxymogroside V (I), deoxymogroside V (II), 11-O-mogroside V (I), mogroside V isomer, mogroside V, iso-mogroside V, 7-O-mogroside V, 11-O-mogroside VI, mogroside VI (I), mogroside VI (II), mogroside VI (III)(neomogroside) and mogroside VI (IV), and mixtures thereof.

19. The beverage according to claim 16, wherein the sugar alcohols are selected from sorbitol, xylitol, inositol, mannitol, erythritol, and mixtures thereof.
